(19) United States
(12) Patent Application Publication
(10) Pub. No.: US 2025/0239361 A1
(43) Pub. Date: Jul. 24, 2025

(54) USING MULTIPLE DEVICES TO MONITOR PHYSIOLOGICAL DATA

(71) Applicant: MONOVO, LLC, Orem, UT (US)

(72) Inventors: STEVEN TAYLOR, Orem, UT (US); KINNEAR ELLIOT, Orem, UT (US)

(21) Appl. No.: 18/705,458

(22) PCT Filed: Oct. 27, 2022
(86) PCT No.: PCT/US2022/048004

§ 371 Date: Apr. 26, 2024

Related U.S. Application Data
(60) Provisional application No. 63/272,413, filed on Oct. 27, 2021.
(60) Provisional application No. 63/272,428, filed on Oct. 27, 2021.
(60) Provisional application No. 63/272,529, filed on Oct. 27, 2021.
(60) Provisional application No. 63/272,545, filed on Oct. 27, 2021.
(60) Provisional application No. 63/272,552, filed on Oct. 27, 2021.
(60) Provisional application No. 63/272,553, filed on Oct. 27, 2021.
(60) Provisional application No. 63/272,431, filed on Oct. 27, 2021.

(51) Int. Cl. G16H 40/67; G16H 50/30

(52) U.S. Cl. CPC G16H 40/67; G16H 50/30

(57) ABSTRACT

A method for using a plurality of wearable devices to monitor physiological data includes receiving one or more first sets of physiological data from a first wearable device that is placed at a first location of a wearer body and receiving one or more second sets of physiological data from a second wearable device that is placed at a second location of a wearer body. The method also includes generating one or more overall values of one or more physiological metrics based on the one or more first sets of physiological data and the one or more second sets of physiological data and sending the one or more overall values of the one or more physiological metrics to a cloud service.

### CROSS-REFERENCE TO RELATED APPLICATION

This application claims priority to and the benefit of U.S. Provisional Patent Application Nos. 63/272,529, filed Oct. 27, 2021; 63/272,413, filed Oct. 27, 2021; 63/272,545, filed Oct. 27, 2021; 63/272,552, filed Oct. 27, 2021; 63/272,553, filed Oct. 27, 2021; and 63/272,428, filed Oct. 27, 2021. Each of the foregoing applications is incorporated herein by reference in its entirety.

### BACKGROUND

Heart rate may be collected in different ways via different types of sensors, such as electrocardiogramansors and photoplethysmogram (PPG) sensors. However, different types of sensors have their own limitations, and the accuracy of different heart rate sensors can be spread out in a wide spectrum depending on the user and the location where the sensors are placed.

The subject matter claimed herein is not limited to embodiments that solve any disadvantages or that operate only in environments such as those described above. Rather, this background is only provided to illustrate one exemplary technology area where some embodiments described herein may be practiced.

### BRIEF SUMMARY

The embodiments described herein are related to a method, a computer system, and/or a computer program product for using a plurality of devices to monitor physiological data. A first wearable device among the plurality of devices is placed at a first location of a wearer's body. A first value of a physiological metric is detected using the first wearable device. A second wearable device among the plurality of devices is placed at a second location of a wearer's body. A second value of the physiological metric is detected using the second wearable device.

The computer system is configured to receive the first value of the physiological metric from the first wearable device, receive the second value of the physiological metric from the second wearable device, and generate an overall value of the physiological metric based on the first value and the second value. The first value, the second value, and/or the overall value of the physiological metric and/or are sent to a cloud service.

In some embodiments, the computer system is further configured to determine whether the first value and the second value are consistent with each other. In response to determining that the first value and the second value are consistent with each other, the first value, the second value, or the overall value of the physiological metric is sent to the cloud service. In response to determining that the first value and the second value are inconsistent with each other, the computer system refrains from sending the first value, the second value, or the overall value of the physiological metric to the cloud service.

Additional features and advantages will be set forth in the description which follows, and in part will be obvious from the description, or may be learned by the practice of the teachings herein. Features and advantages of the invention may be realized and obtained by means of the instruments and combinations particularly pointed out in the appended claims. Features of the present invention will become more fully apparent from the following description and appended claims or may be learned by the practice of the invention as set forth hereinafter.

### BRIEF DESCRIPTION OF THE DRAWINGS

In order to describe the manner in which the above-recited and other advantages and features can be obtained, a more particular description of the subject matter briefly described above will be rendered by reference to specific embodiments which are illustrated in the appended drawings. Understanding that these drawings depict only typical embodiments and are not, therefore, to be considered to be limiting in scope, embodiments will be described and explained with additional specificity and details through the use of the accompanying drawings in which:

FIG. 1 illustrates an example of an environment in which the principles described herein may be implemented;

FIG. 2 illustrates an example architecture of wearable devices and a mobile device, in which the principles described herein may be implemented;

FIG. 3 illustrates an example of a process for training a machine learning AI model for identifying correlations between the heart rates, body temperature, and respiratory rates;

FIG. 4 illustrates a flowchart of an example method for using devices to monitor physiological data; and

FIG. 5 illustrates a flowchart of an example method for determining reliability scores for each set of physiological data.

### DETAILED DESCRIPTION

The embodiments described herein are related to a method, a computer system, and/or a computer program product for using a plurality of devices to monitor physiological data.

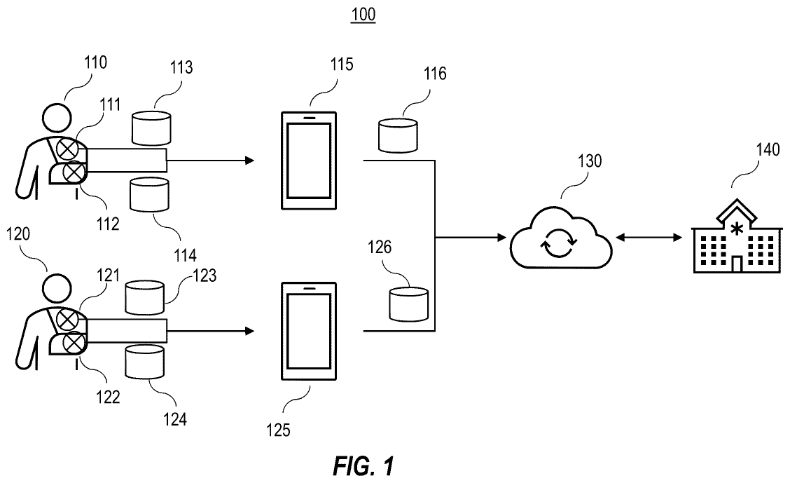

FIG. 1 illustrates an environment 100 in which the principles described herein can be implemented. As illustrated, a first wearer 110 wears a first wearable device 111 and a second wearable device 112 simultaneously. Each of the first wearable device 111 and the second wearable device 112 is configured to generate physiological data. The first wearable device 111 is placed at a first location of the body of the first wearer 110. The second wearable device 112 is placed at a second location of the body of the first wearer 110. The first wearable device 111 and the second wearable device 112 may be the same type or different types of wearable devices. The first wearable device 111 is configured to generate a first set of physiological data 113 of the wearer 110, and the second wearable device 111 is configured to generate a second set of physiological data 114 of the wearer 110. Both the first set of physiological data 113 and the second set of physiological data 114 are sent to a mobile device 115. The mobile device 115 may be a mobile device of the wearer 110, a mobile device of a family member of the wearer 110, and/or a healthcare provider of the wearer 110.

The first set of physiological data 113 and the second set of physiological data 141 complement each other; thus, such data are also collectively referred to as complementary data. After receiving the first set of physiological data 113 and the second set of physiological data 114, the mobile device 115 is configured to use the complementary data sets to generate an overall set of physiological data 116, and send the overall set of physiological data 116 to a cloud service 130. The cloud service 130 may then be accessed via a wearer's portal, a hospital 140's portal, a health care provider's portal, and/or a family member's portal.

For example, in some embodiments, the first set of physiological data 113 is a first value of a physiological metric (e.g., a heart rate, a heart rate variability, and/or a respiratory rate), and the second set of physiological data 114 is a second value of a physiological metric (e.g., a heart rate, a heart rate variability, and/or a respiratory rate) generated by sensors positioned at different locations of the wearer's body. In some embodiments, in response to receiving the first value and the second value of the physiological metric, the mobile device 115 is configured to determine whether the first value and the second value are consistent with each other. In response to determining that the first value and the second value are consistent with each other, the mobile device 115 generates an overall value of the physiological metric and sends the overall value of the physiological metric to the cloud service 130.

The overall value of the physiological metric may be generated based on a weighted average, or based on any predetermined algorithm. For example, if the chest sensor is generally more accurate than a wrist sensor, a greater weight may be given to the value of the physiological metric generated by the chest sensor. In some embodiments, the mobile device 115 may simply randomly select one of the first value 113 or the second value 114 and send the randomly selected value to the cloud service 130.

Notably, there is also a second wearer 120 in the environment 100. Similar to the first wearer 110, the second wearer 120 also wears a first wearable device 121 and a second wearable device 122 simultaneously. The first wearable device 121 is configured to generate a first set of physiological data 123, and the second wearable device 122 is configured to generate a second set of physiological data 124. Both the first wearable device 121 and the second wearable device 122 are configured to send the first set of physiological data 123 and the second set of physiological data 124 to a second mobile device 125. The second mobile device 125 may be a mobile device of the wearer 120, a mobile device of a family member of the wearer 120, and/or a mobile device of a healthcare provider. The second mobile device 125 is configured to receive the first set of physiological data 123 and the second set of physiological data 124 to generate an aggregated physiological data 126, and send the aggregated physiological data 126 to the cloud service 130.

Even though in FIG. 1, there are only two wearers 110, 120 shown in the environment 100, there may be any number of wearers that are simultaneously wearing wearable devices that send physiological data to their mobile devices, which in turn send aggregated physiological data to the cloud service.

Also, as illustrated in FIG. 1, each wearer 110, 120 only wears two wearable devices. However, it is not necessary that each wearer 110, 120 only wears two wearable devices. The more devices the wearer 110, 120 is wearing, the more complementary data would be received, and the mobile device 115, 125 would be able to more quickly and/or more accurately derive the wearer physiological metric 116, 126. For example, the wearer 110 may be wearing 5 different heart rate sensors, one of which is on the wearer's chest, one of which is on the wearer's left wrist, one of which is on the wearer's right wrist, one of which is on the wearer's left ankle, and one of which is on the wearer's right ankle. When 5 sensors are worn, the mobile device 115 is configured to receive 5 sets of physiological data. In some embodiments, the mobile device 115 may require that at least three sets of physiological data are consistent with each other. When three sets of physiological data are consistent with each other, and the other two sets of physiological data are inconsistent with each other, the inconsistent data may be discarded, and the consistent data may be aggregated to generate an overall physiological metric 116.

Further, depending on circumstances, in some cases, the health care provider needs the physiological metric to be more accurate; and in some other cases, the health care provider does not need the physiological metric to be as accurate. A different number of wearable devices may be worn by the wearer 110, 120 depending on the accuracy needs. Also, the threshold for aggregating the multiple datasets and sending the aggregated datasets to the cloud service 130 may also depend on the accuracy needs. For example, when a wearer is in critical condition, more accurate data might be required. When a wearer is fairly healthy, less accurate data might be sufficient.

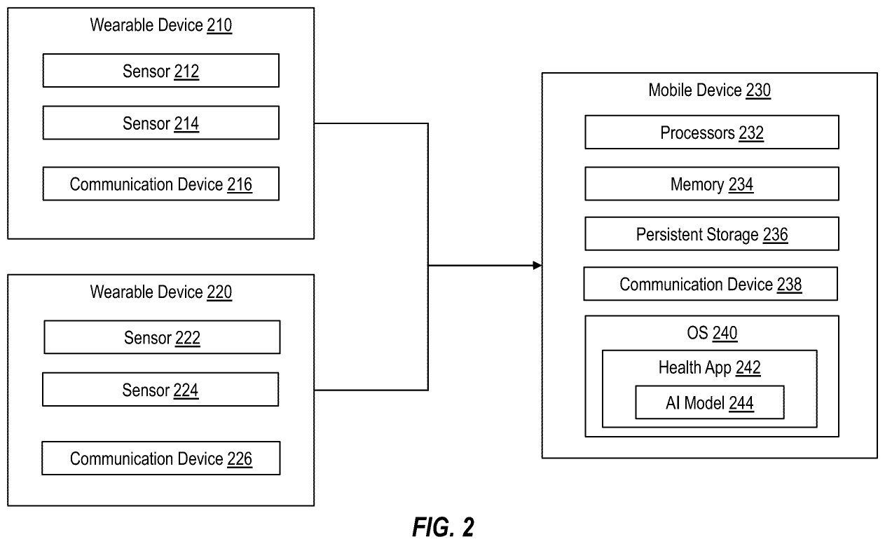

FIG. 2 illustrates an example of an architecture of the wearable devices 210, 220 (which corresponds to the wearable devices 111, 112, 121, 122 of FIG. 1) and a mobile device 230 (which corresponds to the mobile device 115, 125 of FIG. 1). As illustrated in FIG. 2, the wearable device 210 includes a first sensor 212 and a second sensor 214, and a communication device 216. In some embodiments, the first sensor 212 and the second sensor 214 are two different types of sensors. In some embodiments, the plurality of types of sensors includes two or more of the following: (1) an electrocardiography (ECG) heart rate sensor, (2) an photoplethysmogram (PPG) heart rate sensor, (3) a thermometer, and (4) an accelerometer. For example, the first sensor 212 may be an electrocardiography (ECG) sensor, and the second sensor 214 may be a photoplethysmogram (PPG) sensor. The ECG sensor detects electrical signals and determines a heart rate of a wearer based on the detected electrical signals. The PPG detects light signals and determines a heart rate of a wearer based on detected light signals. The ECG sensor and the PPG sensor are two different types of sensors configured to detect a same physiological metric.

In some embodiments, the first sensor 212 and the second sensor 214 are configured to detect different physiological metrics that are related to each other. For example, the first sensor 212 may be an ECG or PPG sensor configured to detect a heart rate. The second sensor 214 may be a temperature sensor and/or a respiratory sensor. When a wearer's temperature changes, the wearer's respiratory rate, and heart rate often change accordingly. As another example, when the wearer's heart rate varies, the wearer's respiratory rate often changes accordingly. In such cases, receiving the multiple sets of physiological data, the mobile device 230 may be configured to correlate the different datasets.

Alternatively, or in addition, in some embodiments, when one set of data fluctuates, the other set of data is also likely to fluctuate. When the fluctuations of the two sets of data are consistent with each other, the mobile device may determine that the received physiological metrics are reliable. When the fluctuations of the two sets of data are inconsistent with each other, the mobile device may determine that the received physiological metric are not reliable.

Further, as illustrated, the wearable device 210 also includes a communication device 216 configured to transmit the physiological data to the mobile device 230. The communication device 216 may be a Bluetooth interface, a WiFi interface, an NFC interface, a 3G, 4G, and/or 5G interface. In some embodiments, the communication device 216 is a Bluetooth interface configured to be paired with a wearer's mobile device 230. Once the two devices are paired, the wearable device 210 is configured to transmit physiological data generated by the first sensor 212 and the second sensor 214 to the mobile device 230 via a Bluetooth connection.

The wearable device 220 is similar to the wearable device 210. The wearable device 220 also has a first sensor 222, a second sensor 224, and a communication device 226. However, the wearable devices 210 and 220 may be the same or different types of devices. Regardless of whether the wearable devices 210, 220 are the same or different types of devices, the mobile device 230 is configured to receive and analyze physiological data generated by the wearable devices 210, 220. Again, even though only two wearable devices 210, 220 are shown in FIG. 2, there may be more than 2 devices simultaneously connected to the mobile device 230.

The mobile device 230 includes one or more processors 232, one or more memories 234, one or more persistent storage devices 236, and one or more communication devices 238. At least one of the communication devices 238 is configured to receive physiological data generated by the wearable devices 210, 220. At least one of the communication devices 238 is configured to send the aggregated data to a cloud service.

The mobile device 230 also includes an operating system 240, which is stored on the persistent storage device(s) 236 and/or loaded in the memory 234. Further, a health application 242 may be installed on the operating system configured to analyze the physiological data received from the wearable devices 210, 220. In some embodiments, the health application 242 includes an AI model, which may be a machine-trained AI model configured to identify correlations between different types of physiological data and/or to determine a reliability score of the received physiological data. The processing performed by the AI model may be carried out at the mobile device 230 (which corresponds to mobile devices 115 and 125), as shown, and/or at a cloud service (such as cloud service 130).

Based on the identified correlations, the health app 242 may be configured to determine whether a particular set of physiological data is sufficiently reliable. In some embodiments, the health app 242 may be configured to give a reliability score to each set of the physiological data, and only when the reliability score is greater than a threshold, the corresponding physiological dataset is sent to the cloud service. When the reliability score is no greater than a threshold, the corresponding physiological dataset is discarded.

In some embodiments, receiving the first set of physiological data and the second set of physiological data includes receiving a heart rate generated by the ECG heart rate sensor or the PPG heart rate sensor, receiving a body temperature generated by the thermometer, and receiving a respiratory rate generated by the accelerometer. A machine-trained artificial intelligence (AI) model 244 is used to identify correlations among the heart rate, the body temperature, or the respiratory rate and determine whether the heart rate is consistent with the body temperature or the respiratory rate.

In some embodiments, in response to determining that the heart rate is consistent with the body temperature or the respiratory rate, the heart rate is provided to the cloud service. In some embodiments, the mobile device 230 is further configured to adjust the heart rate to generate an updated heart rate based on the correlations among the heart rate, the body temperature, or the respiratory rate.

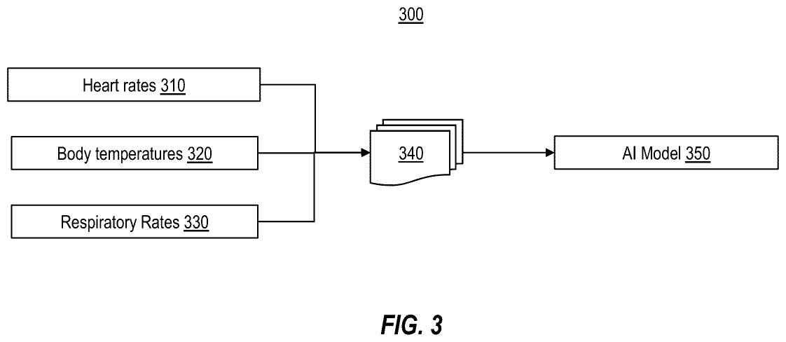

The principles described herein are also related to training a machine-learned AI model to identify correlations among heart rates, body temperatures, or respiratory rates. FIG. 3 illustrates an example of a process 300 for training a machine-learned AI model 350 for identifying correlations between heart rates, body temperatures, and respiratory rates. As illustrated, a plurality of datasets associated with heart rates 310, body temperatures 320, and respiratory rates 330 of a plurality of wearers are used as training data and sent to a machine learning network 340. The machine-learning network 340 may be a neural network or decision trees and/or any other machine learning technologies configured to train the machine-learned AI model to identify correlations among heart rates, body temperature, and/or respiratory rates. Once the AI model 350 is sufficiently trained, the AI model 350 may be deployed onto the health application 242 of the mobile device 230.

The following discussion now refers to a number of methods and method acts that may be performed. Although the method acts may be discussed in a certain order or illustrated in a flow chart as occurring in a particular order, no particular ordering is required unless specifically stated, or required because an act is dependent on another act being completed prior to the act being performed.

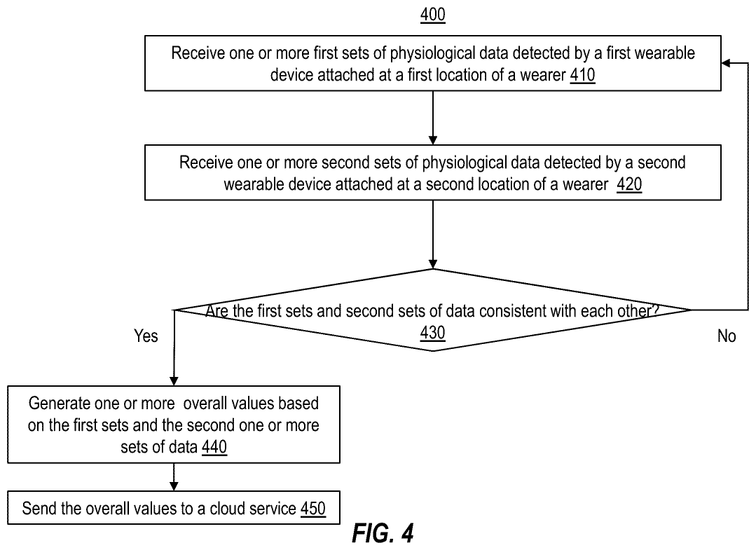

FIG. 4 illustrates a flowchart of an example method 400 for receiving physiological data from complementary wearable devices. The method 400 includes receiving one or more first sets of physiological data detected by a first wearable device attached at a first location of a wearer (act 410). The first wearable device may have multiple sensors, which may be a same type or different types of sensors, and which may be configured to detect a same physiological metric or different physiological metrics. The method 400 further includes receiving one or more second sets of physiological data detected by a second wearable device attached to a second location of the wearer (act 420). The second wearable device may also have multiple sensors, which may be a same type or different types of sensors, and which may be configured to detect a same physiological metric or different physiological metrics. The first wearable device and the second wearable device may be a same type of device or different types of wearable devices, and may be configured to detect a same physiological metric or different physiological metrics.

The method 400 further includes determining whether the one or more first sets of physiological data and the one or more second sets of physiological data are consistent with each other (act 430). The act 430 may be performed via a predetermined algorithm or an AI model deployed on a mobile device that has received the one or more first sets of physiological data and the one or more second sets of physiological data. In response to determining that the one or more first sets of physiological data and the one or more second sets of physiological data are consistent with each other, the mobile device may generate one or more overall values based on the one or more first sets of physiological data and the one or more second sets of physiological data (act 440). For example, the mobile device may be configured to generate an overall value of heart rate, an overall value of temperature, an overall value of respiratory rate, etc. In some embodiments, the one or more overall values are sent to a cloud service (act 450), which can be accessed by a patient portal, a healthcare provider portal, and/or a family member portal, etc.

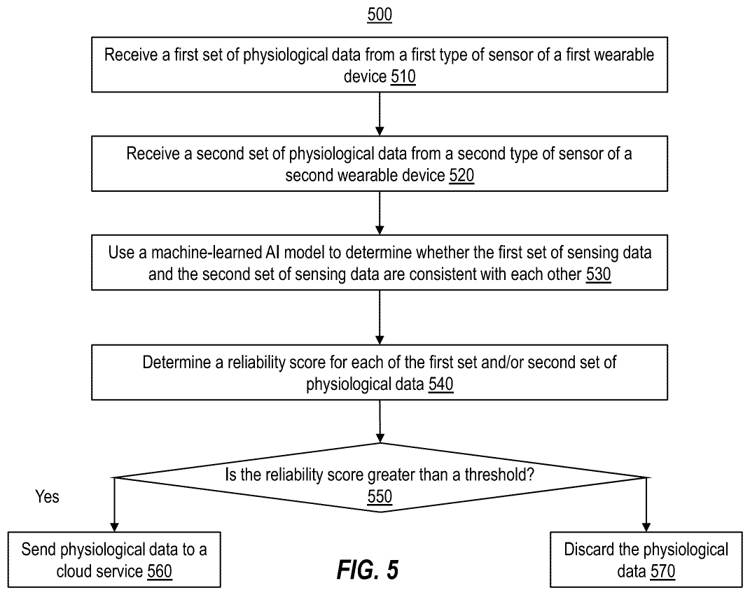

FIG. 5 illustrates a flowchart of an example method 500 for determining a reliability score using a machine-learned AI model. The method 500 includes receiving a first set of physiological data from a first type of sensor of a first wearable device (act 510) and receiving a second set of physiological data from a second type of sensor of a second wearable device (act 520). The first wearable device and the second wearable device may be a same wearable device having two different types of sensors or may be different wearable devices.

The method 500 further includes using a machine-learned AI model to determine whether the first set of physiological data and the second set of physiological data are consistent with each other (act 530) and determining a reliability score for each of the first set of physiological data and/or the second set of physiological data (act 540). The location on the user of the first and second types of sensors may also affect the respective reliability scores for the first and second sets of physiological data. For example, a pulse oximeter placed on an extremity such as a finger will be subject to greater levels of motion and blood flow variation (e.g., during periods of reduced temperature or shock) than a pulse oximeter placed near the core of the body where blood flow is less variable.

The method 500 further includes determining whether the reliability score of the first set of physiological data and/or the second set of physiological data is greater than a threshold (act 550). In response to determining that the reliability score is greater than the threshold, the first set of physiological data and/or the second set of physiological data are sent to a cloud service (act 560). On the other hand, in response to determining that the reliability score is no greater than the threshold, the first set of physiological data and/or the second set of physiological data may be discarded (act 570).

Although the subject matter has been described in language specific to structural features and/or methodological acts, it is to be understood that the subject matter defined in the appended claims is not necessarily limited to the described features or acts described above, or the order of the acts described above. Rather, the described features and acts are disclosed as example forms of implementing the claims.

The present invention may comprise or utilize a special-purpose or general-purpose computer system that comprises computer hardware, such as, for example, one or more processors and system memory, as discussed in greater detail below. Configurations within the scope of the present invention also comprise physical and other computer-readable media for carrying or storing computer-executable instructions and/or data structures. Such computer-readable media can be any available media that can be accessed by a general-purpose or special-purpose computer system. Computer-readable media that store computer-executable instructions and/or data structures are computer storage media. Computer-readable media that carry computer-executable instructions and/or data structures are transmission media. Thus, by way of example, and not limitation, configurations of the invention can comprise at least two distinctly different kinds of computer-readable media: computer storage media and transmission media.

Computer storage media are physical storage media that store computer-executable instructions and/or data structures. Physical storage media comprise computer hardware, such as RAM, ROM, EEPROM, solid state drives (“SSDs”), flash memory, phase-change memory (“PCM”), optical disk storage, magnetic disk storage or other magnetic storage devices, or any other hardware storage device(s) which can be used to store program code in the form of computer-executable instructions or data structures, which can be accessed and executed by a general-purpose or special-purpose computer system to implement the disclosed functionality of the invention.

Transmission media can comprise a network and/or data links which can be used to carry program code in the form of computer-executable instructions or data structures, and which can be accessed by a general-purpose or special-purpose computer system. A “network” is defined as one or more data links that enable the transport of electronic data between computer systems and/or modules and/or other electronic devices. When information is transferred or provided over a network or another communications connection (either hardwired, wireless, or a combination of hardwired or wireless) to a computer system, the computer system may view the connection as transmission media. Combinations of the above should also be comprised within the scope of computer-readable media.

Further, upon reaching various computer system components, program code in the form of computer-executable instructions or data structures can be transferred automatically from transmission media to computer storage media (or vice versa). For example, computer-executable instructions or data structures received over a network or data link can be buffered in RAM within a network interface module (e.g., a “NIM”), and then eventually transferred to computer system RAM and/or to less volatile computer storage media at a computer system. Thus, it should be understood that computer storage media can be comprised in computer system components that also (or even primarily) utilize transmission media.

Computer-executable instructions comprise, for example, instructions and data which, when executed at one or more processors, cause a general-purpose computer system, special-purpose computer system, or special-purpose processing device to perform a certain function or group of functions. Computer-executable instructions may be, for example, binaries, intermediate format instructions such as assembly language, or even source code.

Those skilled in the art will appreciate that the invention may be practiced in network computing environments with many types of computer system configurations, including, personal computers, desktop computers, laptop computers, message processors, hand-held devices, multi-processor systems, microprocessor-based or programmable consumer electronics, network PCs, minicomputers, mainframe computers, mobile telephones, PDAs, tablets, pagers, routers, switches, and the like. The invention may also be practiced in distributed system environments where local and remote computer systems, which are linked (either by hardwired data links, wireless data links, or by a combination of hardwired and wireless data links) through a network, both perform tasks. As such, in a distributed system environment, a computer system may comprise a plurality of constituent computer systems. In a distributed system environment, program modules may be located in both local and remote memory storage devices.

Those skilled in the art will also appreciate that the invention may be practiced in a cloud-computing environment. Cloud computing environments may be distributed, although this is not required. When distributed, cloud computing environments may be distributed internationally within an organization and/or have components possessed across multiple organizations. In this description and the following claims, “cloud computing” is defined as a model for enabling on-demand network access to a shared pool of configurable computing resources (e.g., networks, servers, storage, applications, and services). The definition of “cloud computing” is not limited to any of the other numerous advantages that can be obtained from such a model when properly deployed.

A cloud-computing model can be composed of various characteristics, such as on-demand self-service, broad network access, resource pooling, rapid elasticity, measured service, and so forth. A cloud-computing model may also come in the form of various service models such as, for example, Software as a Service (“SaaS”), Platform as a Service (“PaaS”), and Infrastructure as a Service (“IaaS”). The cloud-computing model may also be deployed using different deployment models such as private cloud, community cloud, public cloud, hybrid cloud, and so forth.

Some configurations, such as a cloud-computing environment, may comprise a system that comprises one or more hosts that are each capable of running one or more virtual machines. During operation, virtual machines emulate an operational computing system, supporting an operating system and perhaps one or more other applications as well. In some configurations, each host comprises a hypervisor that emulates virtual resources for the virtual machines using physical resources that are abstracted from view of the virtual machines. The hypervisor also provides proper isolation between the virtual machines. Thus, from the perspective of any given virtual machine, the hypervisor provides the illusion that the virtual machine is interfacing with a physical resource, even though the virtual machine only interfaces with the appearance (e.g., a virtual resource) of a physical resource. Examples of physical resources including processing capacity, memory, disk space, network bandwidth, media drives, and so forth.

For the processes and methods disclosed herein, the operations performed in the processes and methods may be implemented in differing order. Furthermore, the outlined operations are only provided as examples, and some of the operations may be optional, combined into fewer steps and operations, supplemented with further operations, or expanded into additional operations without detracting from the essence of the disclosed embodiments.

The present invention may be embodied in other specific forms without departing from its spirit or characteristics. The described embodiments are to be considered in all respects only as illustrative and not restrictive. The scope of the invention is, therefore, indicated by the appended claims rather than by the foregoing description. All changes which come within the meaning and range of equivalency of the claims are to be embraced within their scope.

1. A method, implemented at a computer system, for using a plurality of wearable devices to monitor physiological data, comprising:
receiving one or more first sets of physiological data from a first wearable device that is placed at a first location of a wearer body;
receiving one or more second sets of physiological data using a second wearable device that is placed at a second location of the wearer body;
generating one or more overall values of one or more physiological metrics based on the one or more first sets of physiological data and the one or more second sets of physiological data; and
sending the one or more overall values of the one or more physiological metrics to a cloud service.

2. The method of claim 1, the method further comprising:
determining whether the one or more first sets of physiological data and the one or more second sets of physiological data are consistent with each other;
in response to determining that the one or more first sets of physiological data and the one or more second sets of physiological data are consistent with each other, sending one or more overall values of the one or more physiological metrics to the cloud service; and
in response to determining that the one or more first sets of physiological data and the one or more second sets of physiological data are inconsistent with each other, refraining from sending data to the cloud service.

3. The method of claim 1, wherein the physiological metric is at least one of (1) a heart rate of the wearer, (2) a respiratory rate of the wearer, or (3) a temperature of the wearer.

4. The method of claim 1, wherein:
the plurality of wearable devices include a plurality of types of sensors;
receiving a first set of physiological data from a first type of sensor; and
receiving a second set of physiological data from a second type of sensor.

5. The method of claim 1, wherein each of the plurality of wearable devices include a plurality of types of sensors.

6. The method of claim 5, wherein the plurality of types of sensors comprises two of more of the following: (1) an electrocardiography (ECG) heart rate sensor, (2) an photoplethysmogram (PPG) heart rate sensor, (3) a thermometer, and (4) an accelerometer.

7. The method of claim 6, wherein receiving the first set of physiological data and the second set of physiological data includes:
receiving a first heart rate generated by the ECG heart rate sensor;
receiving a second heart rate generated by the PPG heart rate sensor; and
computing an overall heart rate based on the first heart rate and the second heart rate.

8. The method of claim 6, the method further comprising:
receiving a heart rate generated by the ECG heart rate sensor or the PPG heart rate sensor;
receiving a body temperature generated by the thermometer;
receiving a respiratory rate generated by the accelerometer;
using a machine-trained artificial intelligence (AI) model to identify correlations among the heart rate, the body temperature, or the respiratory rate; and
determining whether the heart rate is consistent with the body temperature or the respiratory rate.

9. The method of claim 8, the method further comprising:
in response to determining that the heart rate is consistent with the body temperature or the respiratory rate, providing the heart rate to the cloud service.

10. A method for training a machine-learned AI model to identify correlations among heart rates, body temperatures, or respiratory rates, the method comprising:
obtaining a plurality of datasets associated with heart rates, body temperatures, and respiratory rates of a plurality of wearers;
providing a machine-learning network; and
using the plurality of datasets as training data to train a machine-learned AI model via the machine-learning network,
wherein the machine-learning network is configured to train the machine-learned AI model to identify correlations among heart rates, body temperature, or respiratory rates.

11. The method of claim 10, the method further comprising deploying the machine-learned AI model onto a mobile device.

12. The method of claim 11, the method further comprising deploying the machine-learned AI model onto a wearable device.

13. A mobile computer system comprising:
One or more processors; and
One or more computer-readable hardware storage devices having stored thereon computer-executable instructions that are structured such that, when executed by the one or more processors, the computer-executable instructions configure the mobile computing system to perform the following:
receive one or more first sets of physiological data from a first wearable device that is placed at a first location of a wearer body;
receive one or more second sets of physiological data using a second wearable device that is placed at a second location of the wearer body;
generate one or more overall values of one or more physiological metrics based on the one or more first sets of physiological data and the one or more second sets of physiological data; and
send the one or more overall values of the one or more physiological metrics to a cloud service.

14. The mobile computer system of claim 13, the computer system further configured to:
determine whether the one or more first sets of physiological data and the one or more second sets of physiological data are consistent with the each other;
in response to determining that the one or more first sets of physiological data and the one or more second sets of physiological data are consistent with each other, send one or more overall values of the one or more physiological metrics to the cloud service; and
in response to determining that the one or more first sets of physiological data and the one or more second sets of physiological data are inconsistent with each other, refrain from sending data to the cloud service.

15. The mobile computer system of claim 13, wherein the physiological metric is at least one of (1) a heart rate of the wearer, (2) a respiratory rate of the wearer, or (3) a temperature of the wearer.

16. The mobile computer system of claim 13, wherein:
the plurality of wearable devices include a plurality of types of sensors;
the mobile computer system is further configured to:
receive a first set of physiological data from a first type of sensor; and
receive a second set of physiological data from a second type of sensor.

17. The mobile computer system of claim 16, wherein each of the plurality of wearable devices includes a plurality of types of sensors.

18. The mobile computer system of claim 16, wherein the plurality of types of sensors comprises two or more of the following: (1) an electrocardiography (ECG) heart rate sensor, (2) an photoplethysmogram (PPG) heart rate sensor, (3) a thermometer, and (4) an accelerometer.

19. The mobile computer system of claim 18, wherein receiving the first set of physiological data and the second set of physiological data includes:
receiving a first heart rate generated by the ECG heart rate sensor;
receiving a second heart rate generated by the PPG heart rate sensor; and
computing an overall heart rate based on the first heart rate and the second heart rate.

20. The mobile computer system of claim 19, the mobile computer system further configured to:
receiving a heart rate generated by the ECG heart rate sensor or the PPG heart rate sensor;
receiving a body temperature generated by the thermometer;
receiving a respiratory rate generated by the accelerometer;
using a machine-trained artificial intelligence (AI) model to identify correlations among the heart rate, the body temperature, or the respiratory rate; and
determining whether the heart rate is consistent with the body temperature or the respiratory rate.
